(19) United States
(12) Patent Application Publication
(10) Pub. No.: US 2003/0045683 A1
(43) Pub. Date: Mar. 6, 2003

(54) H14 DNA and polypeptides

(75) Inventors: David John Cosman, Bainbridge Island, WA (US); Bruce Mosley, Seattle, WA (US)

Correspondence Address: IMMUNEX CORPORATION, LAW DEPARTMENT, 51 UNIVERSITY STREET, SEATTLE, WA, 98101

(73) Assignee: Immunex Corporation

(21) Appl. No.: 10/056,984

(22) Filed: Jan. 25, 2002

Related U.S. Application Data
(63) Continuation of application No. 09/392,746, filed on Sep. 9, 1999, now abandoned.
Application No. 09/392,746 is a continuation of application No. PCT/US99/00516, filed on Jan. 8, 1999.
(60) Provisional application No. 60/070,885, filed on Jan. 9, 1998.

(51) Int. Cl. C07K 14/74; C07H 21/04; C12P 21/02; C12N 5/06

(52) U.S. Cl. 530/350; 435/69.7; 435/320.1; 435/325; 536/23.5

(57) ABSTRACT

DNA encoding H14 polypeptides and methods for using the encoded polypeptides are disclosed.

### CROSS-REFERENCE TO RELATED APPLICATIONS

This application is a continuation of U.S. application Ser. No. 09/392,746, filed Sep. 9, 1999; which is a continuation application under 35 § U.S.C. 111(a) of International Application No. PCT/US99/00516, filed Jan. 8, 1999 and published in English on Jul. 15, 1999; which claims the benefit of U.S. Provisional Application Ser. No. 60/070,885, filed Jan. 9, 1998; all of which are hereby incorporated by reference.

### FIELD OF THE INVENTION

The invention is directed to purified and isolated H14 polypeptides, the nucleic acids encoding such polypeptides, processes for production of recombinant forms of such polypeptides, antibodies generated against these polypeptides, fragmented peptides derived from these polypeptides, the use of such polypeptides and fragmented peptides as molecular weight markers, the use of such polypeptides and fragmented peptides as controls for peptide fragmentation, and kits comprising these reagents.

### BACKGROUND OF THE INVENTION

The discovery and identification of proteins is at the forefront of modern molecular biology and biochemistry. The identification of the primary structure, or sequence, of a sample protein is the culmination of an arduous process of experimentation. In order to identify an unknown sample protein, the investigator can rely upon comparison of the unknown sample protein to known peptides using a variety of techniques known to those skilled in the art. For instance, proteins are routinely analyzed using techniques such as electrophoresis, sedimentation, chromatography, and mass spectrometry.

Comparison of an unknown protein sample to polypeptides of known molecular weight allows a determination of the apparent molecular weight of the unknown protein sample (T. D. Brock and M. T. Madigan, Biology of Microorganisms 76-77 (Prentice Hall, 6d ed. 1991)). Protein molecular weight standards are commercially available to assist in the estimation of molecular weights of unknown protein samples (New England Biolabs Inc. Catalog:130-131, 1995; J. L. Hartley, U.S. Pat. No. 5,449,758). However, the molecular weight standards may not correspond closely enough in size to the unknown sample protein to allow an accurate estimation of apparent molecular weight.

The difficulty in estimation of molecular weight is compounded in the case of proteins that are subjected to fragmentation by chemical or enzymatic means (A. L. Lehninger, Biochemistry 106-108 (Worth Books, 2d ed. 1981)). Chemical fragmentation can be achieved by incubation of a protein with a chemical, such as cyanogen bromide, which leads to cleavage of the peptide bond on the carboxyl side of methionine residues (E. Gross, Methods in Enz. 11:238-255, 1967). Enzymatic fragmentation of a protein can be achieved by incubation of a protein with a protease that cleaves at multiple amino acid residues (D. W. Cleveland et al., J. Biol. Chem. 252:1102-1106, 1977). Enzymatic fragmentation of a protein can also be achieved by incubation of a protein with a protease, such as Achromobacter protease I (F. Sakiyama and A. Nakata, U.S. Pat. No. 5,248,599; T. Masaki et al., Biochim. Biophys. Acta 660:44-50, 1981; T. Masaki et al., Biochim. Biophys. Acta 660:51-55, 1981), which leads to cleavage of the peptide bond on the carboxyl side of lysine residues. The molecular weights of the fragmented peptides can cover a large range of molecular weights and the peptides can be numerous. Variations in the degree of fragmentation can also be accomplished (D. W. Cleveland et al., J. Biol. Chem. 252:1102-1106, 1977).

The unique nature of the composition of a protein with regard to its specific amino acid constituents results in a unique positioning of cleavage sites within the protein. Specific fragmentation of a protein by chemical or enzymatic cleavage results in a unique “peptide fingerprint” (D. W. Cleveland et al., J. Biol. Chem. 252:1102-1106, 1977; M. Brown et al., J. Gen. Virol. 50:309-316, 1980). Consequently, cleavage at specific sites results in reproducible fragmentation of a given protein into peptides of precise molecular weights. Furthermore, these peptides possess unique charge characteristics that determine the isoelectric pH of the peptide. These unique characteristics can be exploited using a variety of electrophoretic and other techniques (T. D. Brock and M. T. Madigan, Biology of Microorganisms 76-77 (Prentice Hall, 6d ed. 1991)).

When a peptide fingerprint of an unknown protein is obtained, this can be compared to a database of known proteins to assist in the identification of the unknown protein (W. J. Henzel et al., Proc. Natl. Acad. Sci. USA 90:5011-5015, 1993; B. Thiede et al., Electrophoresis 1996, 17:588-599, 1996). A variety of computer software programs are accessible via the Internet to the skilled artisan for the facilitation of such comparisons, such as MultiIdent (Internet site: expasy.ch/sprot/multiident.html), PeptideSearch (Internet site: mann.embl-heiedelberg.de/deSearch/FR_PeptideSearchForm.html), and ProFound (Internet site: chait-sgi.rockefeller.edu/cgi-bin/prot-id-frag.html). These programs allow the user to specify the cleavage agent and the molecular weights of the fragmented peptides within a designated tolerance. The programs compare these molecular weights to protein databases to assist in the elucidation of the identity of the sample protein. Accurate information concerning the number of fragmented peptides and the precise molecular weight of those peptides is required for accurate identification. Therefore, increasing the accuracy in the determination of the number of fragmented peptides and the precise molecular weight of those peptides should result in enhanced success in the identification of unknown proteins.

Fragmentation of proteins is further employed for the production of fragments for amino acid composition analysis and protein sequencing (P. Matsudiara, J. Biol. Chem. 262:10035-10038, 1987; C. Eckerskorn et al., Electrophoresis 1988, 9:830-838, 1988), particularly the production of fragments from proteins with a “blocked” N-terminus. In addition, fragmentation of proteins can be used in the preparation of peptides for mass spectrometry (W. J. Henzel et al., Proc. Natl. Acad. Sci. USA 90:5011-5015, 1993; B. Thiede et al., Electrophoresis 1996, 17:588-599, 1996), for immunization, for affinity selection (R. A. Brown, U.S. Pat. No. 5,151,412), for determination of modification sites (e.g. phosphorylation), for generation of active biological compounds (T. D. Brock and M. T. Madigan, Biology of Microorganisms 300-301 (Prentice Hall, 6d ed. 1991)), and for differentiation of homologous proteins (M. Brown et al., J. Gen. Virol. 50:309-316, 1980).

In view of the continuing interest in protein research and the elucidation of protein structure and properties, there exists a need in the art for polypeptides suitable for use in peptide fragmentation studies and in molecular weight measurements.

### SUMMARY OF THE INVENTION

The invention aids in fulfilling this need in the art. The invention encompasses an isolated nucleic acid molecule comprising the DNA sequence of SEQ ID NO: 1 and an isolated nucleic acid molecule encoding the amino acid sequence of SEQ ID NO: 2. The invention also encompasses nucleic acid molecules complementary to these sequences. As such, the invention includes double-stranded nucleic acid sequences comprising the DNA sequence of SEQ ID NO: 1 and isolated nucleic acid molecules encoding the amino acid sequence of SEQ ID NO: 2. Both single-stranded and double-stranded RNA and DNA H14 nucleic acid molecules are encompassed by the invention. These molecules can be used to detect both single-stranded and double-stranded RNA and DNA variants of H14 encompassed by the invention. A double-stranded DNA probe allows the detection of nucleic acid molecules equivalent to either strand of the nucleic acid molecule. Isolated nucleic acid molecules that hybridize to a denatured, double-stranded DNA comprising the DNA sequence of SEQ ID NO: 1 or an isolated nucleic acid molecule encoding the amino acid sequence of SEQ ID NO: 2 under conditions of moderate stringency in 50% formamide and 6×SSC, at 42° C. with washing conditions of 60° C., 0.5×SSC, 0.1% SDS are encompassed by the invention.

The invention further encompasses isolated nucleic acid molecules derived by in vitro mutagenesis from SEQ ID NO: 1. In vitro mutagenesis would include numerous techniques known in the art including, but not limited to, site-directed mutagenesis, random mutagenesis, and in vitro nucleic acid synthesis. The invention also encompasses isolated nucleic acid molecules degenerate from SEQ ID NO: 1 as a result of the genetic code, isolated nucleic acid molecules which are allelic variants of human H14 DNA or a species homolog of H14 DNA. The invention also encompasses recombinant vectors that direct the expression of these nucleic acid molecules and host cells transformed or transfected with these vectors.

The invention also encompasses isolated polypeptides encoded by these nucleic acid molecules, including isolated polypeptides having a molecular weight of approximately 47 kD as determined by SDS-PAGE and isolated polypeptides in non-glycosylated form. Isolated polyclonal or monoclonal antibodies that bind to these polypeptides are encompassed by the invention. The invention further encompasses methods for the production of H14 polypeptides including culturing a host cell under conditions promoting expression and recovering the polypeptide from the culture medium. Especially, the expression of H14 polypeptides in bacteria, yeast, plant, and animal cells is encompassed by the invention.

In addition, assays utilizing H14 polypeptides to screen for potential inhibitors of activity associated with H14 polypeptide counter-structure molecules, and methods of using H14 polypeptides as therapeutic agents for the treatment of diseases mediated by H14 polypeptide counter-structure molecules are encompassed by the invention. Further, methods of using H14 polypeptides in the design of inhibitors thereof are also an aspect of the invention.

The invention further encompasses the fragmented peptides produced from H14 polypeptides by chemical or enzymatic treatment. In addition, forms of H14 polypeptide molecular weight markers and fragmented peptides thereof, wherein at least one of the sites necessary for fragmentation by chemical or enzymatic means has been mutated, are an aspect of the invention.

The invention also encompasses a method for the visualization of H14 polypeptide molecular weight markers and fragmented peptides thereof using electrophoresis. The invention further includes a method for using H14 polypeptide molecular weight markers and fragmented peptides thereof as molecular weight markers that allow the estimation of the molecular weight of a protein or a fragmented protein sample. The invention further encompasses methods for using H14 polypeptides and fragmented peptides thereof as markers, which aid in the determination of the isoelectric point of a sample protein. The invention also encompasses methods for using H14 polypeptides and fragmented peptides thereof as controls for establishing the extent of fragmentation of a protein sample.

Further encompassed by this invention are kits to aid the determination of molecular weights of a sample protein utilizing H14 polypeptide molecular weight markers, fragmented peptides thereof, and forms of H14 polypeptide molecular weight markers, wherein at least one of the sites necessary for fragmentation by chemical or enzymatic means has been mutated.

### DETAILED DESCRIPTION OF THE INVENTION

A cDNA encoding human H14 polypeptide has been isolated and is disclosed in SEQ ID

| | |
|---|---|
| ATGCCCGCCGGCCGCCGGGGCCCCGCCGCCCAATCCG | (SEQ ID NO:1) |
| CGCGGCGGCCGCCGCCGTTGCTGCCCCTGCTGCTGCT | |
| GCTCTGCGTCCTCGGGGCGCCGCGAGCCGGATCAGGA | |
| GCCCACACAGCTGTGATCAGTCCCCAGGATCCCACGC | |
| TTCTCATCGGCTCCTCCCTGCTGGCCACCTGCTCAGT | |
| GCACGGAGACCCACCAGGAGCCACCGCCGAGGGCCTC | |
| TACTGGACCCTCAACGGGCGCCGCCTGCCCCCTGAGC | |
| TCTCCCGTGTACTCAACGCCTCCACCTTGGCTCTGGC | |
| CCTGGCCAACCTCAATGGGTCCAGGCAGCGGTCGGGG | |
| GACAACCTCGTGTGCCACGCCCGTGACGGCAGCATCC | |
| TGGCTGGCTCCTGCCTCTATGTTGGCCTGCCCCCAGA | |
| GAAACCCGTCAACATCAGCTGCTGGTCCAAGAACATG | |
| AAGGACTTGACCTGCCGCTGGACGCCAGGGGCCCACG | |
| GGGAGACCTTCCTCCACACCAACTACTCCCTCAAGTA | |
| CAAGCTTAGGTGGTATGGCCAGGACAACACATGTGAG | |
| GAGTACCACACAGTGGGGCCCCACTCCTGCCACATCC | |
| CCAAGGACCTGGCTCTCTTTACGCCCTATGAGATCTG | |
| GGTGGAGGCCACCAACCGCCTGGGCTCTGCCCGCTCC | |
| GATGTACTCACGCTGGATATCCTGGATGTGGTGACCA | |
| CGGACCCCCCGCCCGACGTGCACGTGAGCCGCGTCGG | |
| GGGCCTGGAGGACCAGCTGAGCGTGCGCTGGGTGTCG | |
| CCACCCGCCCTCAAGGATTTCCTCTTTCAAGCCAAAT | |
| ACCAGATCCGCTACCGAGTGGAGGACAGTGTGGACTG | |
| GAAGGTGGTGGACGATGTGAGCAACCAGACCTCCTGC | |
| CGCCTGGCCGGCCTGAAACCCGGCACCGTGTACTTCG | |
| TGCAAGTGCGCTGCAACCCCTTTGGCATCTATGGCTC | |
| CAAGAAAGCCGGGATCTGGAGTGAGTGGAGCCACCCC | |
| ACAGCCGCCTCCACTCCCCGCAGTGAGCGCCCGGGCC | |
| CGGGCGGCGGGGCGTGCGAACCGCGGGGCGGAGAGCC | |
| GAGCTCGGGGCCGGTGCGGCGCGAGCTCAAGCAGTTC | |
| CTGGGCTGGCTCAAGAAGCACGCGTACTGCTCCAACC | |
| TCAGCTTCCGCCTCTACGACCAGTGGCGAGCCTGGAT | |
| GCAGAAGTCGCACAAGACCCGCAACCAGCACAGGACG | |
| AGGGGATCCTGCCCTCGGGCAGACGGGGCACGGCGAG | |
| AGGTCCTGCCAGATAAGCTGTAG | |

This discovery of the cDNA encoding human H14 polypeptide enables construction of expression vectors comprising nucleic acid sequences encoding H14 polypeptides; host cells transfected or transformed with the expression vectors; biologically active human H14 polypeptide and H14 molecular weight markers as isolated and purified proteins; and antibodies immunoreactive with H14 polypeptides.

EST data base entry H14009 was proposed as a possible member of the Hematopoietin Receptor Superfamily (HPR) based on analysis of EST database sequences. This family of cytokine receptors is defined by a number of amino acid structures within a 200 amino acid “Hematopoietin Receptor Domain”. The original 218 base pair sequence in the EST database was incorrect, and this EST was not recognized or annotated as encoding a protein related to Hematopoietin receptors. The EST clone was obtained, and by careful sequencing, the original sequencing errors were corrected and a potential hematopoietin receptor family member was then identified. The EST clone was found to encode a polypeptide comprising a portion of the receptor.

An H14 DNA specific single strand probe was used to screen a Dermal Fibroblast cDNA library. Clones isolated from this library encoded 4 different protein sequences each differing at the carboxy terminal end of the molecule. Differences were due to differential DNA splicing (insertions or deletions).

A composite sequence for H14 DNA encodes a soluble hematopoietin receptor with high homology to known hematopoietin receptors, EBI3, CNTF Receptor, IL-6 receptor, and the p40 subunit of IL-12.

H14 polypeptide (SEQ ID NO: 2) is most homologous to a group of hematopoietin receptors that are naturally soluble molecules or are easily released from the cell membrane under natural, physiological conditions in biologically active forms. These include EBI3 (29.0% amino acid identity), Ciliary Neutotrophic Factor (27.9%), IL-6 receptor (25.1%), and the P40 subunit of IL-12 (21.9%).

The sequence of EST clone H14009 overlaps with nucleotides of H14 DNA. Nucleotides 1-1278 of H14 DNA (SEQ ID NO: 1) encode H14 polypeptide (SEQ ID NO: 2).

| | |
|---|---|
| MPAGRRGPAA QSARRPPPLL PLLLLLCVLG | (SEQ ID NO:2) |
| APRAGSGAHT AVISPQDPTL LIGSSLLATC | |
| SVHGDPPGAT AEGLYWTLNG RRLPPELSRV | |
| LNASTLALAL ANLNGSRQRS GDNLVCHARD | |
| GSILAGSCLY VGLPPEKPVN ISCWSKNMKD | |
| LTCRWTPGAH GETFLHTNYS LKYKLRWYGQ | |
| DNTCEEYHTV GPHSCHIPKD LALFTPYEIW | |
| VEATNRLGSA RSDVLTLDIL DVVTTDPPPD | |
| VHVSRVGGLE DQLSVRWVSP PALKDFLFQA | |
| KYQIRYRVED SVDWKVVDDV SNQTSCRLAG | |
| LKPGTVYFVQ VRCNPFGIYG SKKAGIWSEW | |
| SHPTAASTPR SERPGPGGGA CEPRGGEPSS | |
| GPVRRELKQF LGWLKKHAYC SNLSFRLYDQ | |
| WRAWMQKSHK TRNQHRTRGS CPRADGARRE | |
| VLPDKL | |

The signal sequence cleavage is predicted after glycine at position 30 of SEQ ID NO: 2. The Ig-like domain comprises from approximately amino acids 31 or 37 to 134. The hematopoietin receptor-like domain comprises from approximately amino acids 135 to 345.

Northern blot analysis has shown that H14 mRNA is easily detectable in hepatic cells, fibroblasts, T and B cell lines, neural cells, placenta and others.

H14 polypeptide was expressed as the natural form and as an Fc fusion protein utilizing a heterologous CMV leader. The amino acid sequence of CMVL-H14Fc (SEQ ID NO: 3) is:

MARRLWILSL LAVTLTVALA APSQKSKRRT SGAHTAVISP
QDPTLLIGSS LLATCSVHGD PPGATAEGLY WTLNGRRLPP
ELSRVLNAST LALALANLNG SRQRSGDNLV CHARDGSILA
GSCLYVGLPP EKPVNISCWS KNMKDLTCRW TPGAHGETFL
HTNYSLKYKL RWYGQDNTCE EYHTVGPHSC HIPKDLALFT
PYEIWVEATN RLGSARSDVL TLDILDVVTT DPPPDVHVSR
VGGLEDQLSV RWVSPPALKD FLFQAKYQIR YRVEDSVDWK
VVDDVSNQTS CRLAGLKPGT VYFVQVRCNP FGIYGSKKAG
IWSEWSHPTA ASTPRSERPL GYRYVEPRSC DKTHTCPPCP
APEAEGAPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSRDELTK NQVSLTCLVK GFYPSDIAVE WESKGQPENN
YKTTPPVLDS DGYTQKSLSL SPGK

The H14 polypeptide sequence is underlined. Signal sequence cleavage is predicted after alanine at position 20.

Both forms of H14 polypeptide expressed well as soluble molecules on transient expression in CV-1/EBNA cells.

H14 polypeptide is a member of a family of cytokine receptors which, after ligand binding and cell surface localization, induce proliferation and/or differentiation of a wide variety of cell types including those of hematopoietic, hepatic, neural and osteoclastic origin. As with other members of this family, activation of receptor complexes may induce production of other cytokines in certain cell populations.

Antibodies directed against H14 polypeptide or modified versions of this molecule may be used to block specific cell functions elicited through membrane interaction or used to identify or isolate other proteins that specifically interact with the H14 polypeptide.

In one embodiment of this invention, the expression of recombinant H14 polypeptides can be accomplished utilizing fusion of sequences encoding H14 polypeptides to sequences encoding another polypeptide to aid in the purification of H14 polypeptides. An example of such a fusion is a fusion of sequences encoding a H14 polypeptide to sequences encoding the product of the malE gene of the pMAL-c2 vector of New England Biolabs, Inc. Such a fusion allows for affinity purification of the fusion protein, as well as separation of the maltose binding protein portion of the fusion protein from the H14 polypeptide after purification. It is understood of course that many different vectors and techniques can be used for the expression and purification of H14 polypeptides and that this embodiment in no way limits the scope of the invention.

The insertion of DNA encoding the H14 polypeptide into the pMAL-c2 vector can be accomplished in a variety of ways using known molecular biology techniques. The preferred construction of the insertion contains a termination codon adjoining the carboxyl terminal codon of the H14 polypeptide. In addition, the preferred construction of the insertion results in the fusion of the amino terminus of the H14 polypeptide directly to the carboxyl terminus of the Factor Xa cleavage site in the pMAL-c2 vector. A DNA fragment can be generated by PCR using H14 DNA as the template DNA and two oligonucleotide primers. Use of the oligonucleotide primers generates a blunt-ended fragment of DNA that can be isolated by conventional means. This PCR product can be ligated together with pMAL-p2 (digested with the restriction endonuclease Xmn I) using conventional means. Positive clones can be identified by conventional means. Induction of expression and purification of the fusion protein can be performed as per the manufacturer's instructions. This construction facilitates a precise separation of the H14 polypeptide from the fused maltose binding protein utilizing a simple protease treatment as per the manufacturer's instructions. In this manner, purified H14 polypeptide can be obtained. Furthermore, such a constructed vector can be easily modified using known molecular biology techniques to generate additional fusion proteins.

Another preferred embodiment of the invention is the use of H14 polypeptides as molecular weight markers to estimate the apparent molecular weight of a sample protein by gel electrophoresis. An isolated and purified H14 polypeptide molecular weight marker according to the invention has a molecular weight of approximately 46,896 Daltons, in the absence of glycosylation. The H14 polypeptide, together with a sample protein, can be resolved by denaturing polyacrylamide gel electrophoresis by conventional means (U. K. Laemmli, Nature 227:680-685, 1970) in two separate lanes of a gel containing sodium dodecyl sulfate and a concentration of acrylamide between 10-20%. Proteins on the gel can be visualized using a conventional staining procedure. The H14 polypeptide molecular weight marker can be used as a molecular weight marker in the estimation of the apparent molecular weight of the sample protein. The unique amino acid sequence of H14 (SEQ ID NO: 2) specifies a molecular weight of approximately 46,896 Daltons. Therefore, the H14 polypeptide molecular weight marker serves particularly well as a molecular weight marker for the estimation of the apparent molecular weight of sample proteins that have apparent molecular weights close to 46,896 Daltons. The use of this polypeptide molecular weight marker allows an increased accuracy in the determination of apparent molecular weight of proteins that have apparent molecular weights close to 46,896 Daltons. It is understood of course that many different techniques can be used for the determination of the molecular weight of a sample protein using H14 polypeptides and that this embodiment in no way limits the scope of the invention.

Another preferred embodiment of the invention is the use of H14 fragmented peptide molecular weight markers, generated by chemical fragmentation of H14 polypeptide, as molecular weight markers to estimate the apparent molecular weight of a sample protein by gel electrophoresis. Isolated and purified H14 polypeptide can be treated with cyanogen bromide under conventional conditions that result in fragmentation of the H14 polypeptide molecular weight marker by specific hydrolysis on the carboxyl side of the methionine residues within the H14 polypeptide (E. Gross, Methods in Enz. 11:238-255, 1967). Due to the unique amino acid sequence of the H14 polypeptide, the fragmentation of H14 polypeptide molecular weight markers with cyanogen bromide generates a unique set of H14 fragmented peptide molecular weight markers. The distribution of methionine residues determines the number of amino acids in each peptide and the unique amino acid composition of each peptide determines its molecular weight.

The unique set of H14 fragmented peptide molecular weight markers generated by treatment of H14 polypeptide with cyanogen bromide comprises 3 fragmented peptides of at least 10 amino acids in size. The peptide encoded by amino acids 2-148 of SEQ ID NO: 2 has a molecular weight of approximately 15,190 Daltons. The peptide encoded by amino acids 149-395 of SEQ ID NO: 2 has a molecular weight of approximately 28,014 Daltons. The peptide encoded by amino acids 396-426 of SEQ ID NO: 2 has a molecular weight of approximately 3,596 Daltons. Therefore, cleavage of the H14 polypeptide by chemical treatment with cyanogen bromide generates a unique set of H14 fragmented peptide molecular weight markers. The unique and known amino acid sequence of these H14 fragmented peptides allows the determination of the molecular weight of these fragmented peptide molecular weight markers. In this particular case, H14 fragmented peptide molecular weight markers have molecular weights of approximately 15,190; 28,014; and 3,596 Daltons.

The H14 fragmented peptide molecular weight markers, together with a sample protein, can be resolved by denaturing polyacrylamide gel electrophoresis by conventional means in two separate lanes of a gel containing sodium dodecyl sulfate and a concentration of acrylamide between 10-20%. Proteins on the gel can be visualized using a conventional staining procedure. The H14 fragmented peptide molecular weight markers can be used as molecular weight markers in the estimation of the apparent molecular weight of the sample protein. The unique amino acid sequence of H14 specifies a molecular weight of approximately 15,190; 28,014; and 3,596 Daltons for the H14 fragmented peptide molecular weight markers. Therefore, the H14 fragmented peptide molecular weight markers serve particularly well as a molecular weight markers for the estimation of the apparent molecular weight of sample proteins that have apparent molecular weights close to 15,190; 28,014; or 3,596 Daltons. Consequently, the use of these fragmented peptide molecular weight markers allows an increased accuracy in the determination of apparent molecular weight of proteins that have apparent molecular weights close to 15,190; 28,014; or 3,596 Daltons.

In a further embodiment, the sample protein and the H14 polypeptide can be simultaneously, but separately, treated with cyanogen bromide under conventional conditions that result in fragmentation of the sample protein and the H14 polypeptide by specific hydrolysis on the carboxyl side of the methionine residues within the sample protein and the H14 polypeptide. As described above, the H14 fragmented peptide molecular weight markers generated by cleavage of the H14 polypeptide with cyanogen bromide have molecular weights of approximately 15,190; 28,014; and 3,596 Daltons.

The fragmented peptides from both the H14 polypeptide and the sample protein can be resolved by denaturing polyacrylamide gel electrophoresis by conventional means in two separate lanes of a gel containing sodium dodecyl sulfate and a concentration of acrylamide between 10-20%. Fragmented peptides on the gel can be visualized using a conventional staining procedure. The H14 fragmented peptide molecular weight markers can be used as molecular weight markers in the estimation of the apparent molecular weight of the fragmented proteins derived from the sample protein. As discussed above, the H14 fragmented peptide molecular weight markers serve particularly well as a molecular weight markers for the estimation of the apparent molecular weight of fragmented peptides that have apparent molecular weights close to 15,190; 28,014; or 3,596 Daltons. Consequently, the use of these H14 fragmented peptide molecular weight markers allows an increased accuracy in the determination of apparent molecular weight of fragmented peptides that have apparent molecular weights close to 15,190; 28,014; or 3,596 Daltons. The extent of fragmentation of the H14 polypeptide is further used as a control to determine the conditions expected for complete fragmentation of the sample protein. It is understood of course that many chemicals could be used to fragment H14 polypeptides and that this embodiment in no way limits the scope of the invention.

In another embodiment, unique sets of H14 fragmented peptide molecular weight markers can be generated from H14 polypeptide using enzymes that cleave the polypeptide at specific amino acid residues. Due to the unique nature of the amino acid sequence of the H14 polypeptide, cleavage at different amino acid residues will result in the generation of different sets of fragmented peptide molecular weight markers.

An isolated and purified H14 polypeptide can be treated with Achromobacter protease I under conventional conditions that result in fragmentation of the H14 polypeptide by specific hydrolysis on the carboxyl side of the lysine residues within the H14 polypeptide (T. Masaki et al., Biochim. Biophys. Acta 660:44-50, 1981; T. Masaki et al., Biochim. Biophys. Acta 660:51-55, 1981). Due to the unique amino acid sequence of the H14 polypeptide, the fragmentation of H14 polypeptide molecular weight markers with Achromobacter protease I generates a unique set of H14 fragmented peptide molecular weight markers. The distribution of lysine residues determines the number of amino acids in each peptide and the unique amino acid composition of each peptide determines its molecular weight.

The unique set of H14 fragmented peptide molecular weight markers generated by treatment of H14 polypeptide with Achromobacter protease I comprises 10 fragmented peptides of at least 10 amino acids in size. The generation of 10 fragmented peptides with this enzyme treatment of the H14 polypeptide, compared to 3 fragmented peptides with cyanogen bromide treatment of the H14 polypeptide, clearly illustrates that both the size and number of the fragmented peptide molecular weight markers will vary depending upon the fragmentation treatment utilized to fragment the H14 polypeptide. Both the size and number of these fragments are dictated by the amino acid sequence of the H14 polypeptide.

The peptide encoded by amino acids 1-137 of SEQ ID NO: 2 has a molecular weight of approximately 14,061 Daltons. The peptide encoded by amino acids 150-172 of SEQ ID NO: 2 has a molecular weight of approximately 2,646 Daltons. The peptide encoded by amino acids 175-199 of SEQ ID NO: 2 has a molecular weight of approximately 2,969 Daltons. The peptide encoded by amino acids 200-264 of SEQ ID NO: 2 has a molecular weight of approximately 7,137 Daltons. The peptide encoded by amino acids 272-285 of SEQ ID NO: 2 has a molecular weight of approximately 1,855 Daltons. The peptide encoded by amino acids 286-302 of SEQ ID NO: 2 has a molecular weight of approximately 1,803 Daltons. The peptide encoded by amino acids 313-322 of SEQ ID NO: 2 has a molecular weight of approximately 2,231 Daltons. The peptide encoded by amino acids 324-368 of SEQ ID NO: 2 has a molecular weight of approximately 4,653 Daltons. The peptide encoded by amino acids 377-397 of SEQ ID NO: 2 has a molecular weight of approximately 2,702 Daltons. The peptide encoded by amino acids 401-426 of SEQ ID NO: 2 has a molecular weight of approximately 2,988 Daltons. Therefore, cleavage of the H14 polypeptide by enzymatic treatment with Achromobacter protease I generates a unique set of H14 fragmented peptide molecular weight markers. The unique and known amino acid sequence of these fragmented peptides allows the determination of the molecular weight of these H14 fragmented peptide molecular weight markers. In this particular case, these H14 fragmented peptide molecular weight markers have molecular weights of approximately 14,061; 2,646; 2,969; 7,137; 1,855; 1,803; 2,231; 4,653; 2,702; and 2,988 Daltons.

Once again, the H14 fragmented peptide molecular weight markers, together with a sample protein, can be resolved by denaturing polyacrylamide gel electrophoresis by conventional means in two separate lanes of a gel containing sodium dodecyl sulfate and a concentration of acrylamide between 10-20%. Proteins on the gel can be visualized using a conventional staining procedure. The H14 fragmented peptide molecular weight markers can be used as molecular weight markers in the estimation of the apparent molecular weight of the sample protein. The H14 fragmented peptide molecular weight markers serve particularly well as molecular weight markers for the estimation of the apparent molecular weight of proteins that have apparent molecular weights close to 14,061; 2,646; 2,969; 7,137; 1,855; 1,803; 2,231; 4,653; 2,702; or 2,988 Daltons. The use of these fragmented peptide molecular weight markers allows an increased accuracy in the determination of apparent molecular weight of proteins that have apparent molecular weights close to 14,061; 2,646; 2,969; 7,137; 1,855; 1,803; 2,231; 4,653; 2,702; or 2,988 Daltons.

In another embodiment, the sample protein and the H14 polypeptide can be simultaneously, but separately, treated with Achromobacter protease I under conventional conditions that result in fragmentation of the sample protein and the H14 polypeptide by specific hydrolysis on the carboxyl side of the lysine residues within the sample protein and the H14 polypeptide. The H14 fragmented peptide molecular weight markers and the fragmented peptides derived from the sample protein are resolved by denaturing polyacrylamide gel electrophoresis by conventional means in two separate lanes of a gel containing sodium dodecyl sulfate and a concentration of acrylamide between 10-20%. Fragmented peptides on the gel can be visualized using a conventional staining procedure. The H14 fragmented peptide molecular weight markers can be used as molecular weight markers in the estimation of the apparent molecular weight of the sample protein. The H14 fragmented peptide molecular weight markers serve particularly well as molecular weight markers for the estimation of the apparent molecular weight of fragmented peptides that have apparent molecular weights close to 14,061; 2,646; 2,969; 7,137; 1,855; 1,803; 2,231; 4,653; 2,702; or 2,988 Daltons. The use of these H14 fragmented peptide molecular weight markers allows an increased accuracy in the determination of apparent molecular weight of fragmented peptides that have apparent molecular weights close to 14,061; 2,646; 2,969; 7,137; 1,855; 1,803; 2,231; 4,653; 2,702; or 2,988 Daltons. The extent of fragmentation of the H14 polypeptide is further used as a control to determine the conditions expected for complete fragmentation of the sample protein. It is understood of course that many enzymes could be used to fragment H14 polypeptides and that this embodiment in no way limits the scope of the invention.

In another embodiment, monoclonal and polyclonal antibodies against H14 polypeptides can be generated. Balb/c mice can be injected intraperitoneally on two occasions at 3 week intervals with 10 μg of isolated and purified H14 polypeptide or peptides based on the amino acid sequence of H14 polypeptides in the presence of RIBI adjuvant (RIBI Corp., Hamilton, Mont.). Mouse sera are then assayed by conventional dot blot technique or antibody capture (ABC) to determine which animal is best to fuse. Three weeks later, mice are given an intravenous boost of 3 μg of the H14 polypeptide or peptides, suspended in sterile PBS. Three days later, mice are sacrificed and spleen cells fused with Ag8.653 myeloma cells (ATCC) following established protocols. Briefly, Ag8.653 cells are washed several times in serum-free media and fused to mouse spleen cells at a ratio of three spleen cells to one myeloma cell. The fusing agent is 50% PEG: 10% DMSO (Sigma). Fusion is plated out into twenty 96-well flat bottom plates (Coming) containing HAT supplemented DMEM media and allowed to grow for eight days. Supernatants from resultant hybridomas are collected and added to a 96-well plate for 60 minutes that is first coated with goat anti-mouse Ig. Following washes, ¹²⁵I—H14 polypeptide or peptides are added to each well, incubated for 60 minutes at room temperature, and washed four times. Positive wells can be subsequently detected by autoradiography at −70° C. using Kodak X-Omat S film. Positive clones can be grown in bulk culture and supernatants are subsequently purified over a Protein A column (Pharmacia). It is understood of course that many techniques could be used to generate antibodies against H14 polypeptides and fragmented peptides thereof and that this embodiment in no way limits the scope of the invention.

In another embodiment, antibodies generated against H14 and fragmented peptides thereof can be used in combination with H14 polypeptide or fragmented peptide molecular weight markers to enhance the accuracy in the use of these molecular weight markers to determine the apparent molecular weight and isoelectric point of a sample protein. H14 polypeptide or fragmented peptide molecular weight markers can be mixed with a molar excess of a sample protein and the mixture can be resolved by two dimensional electrophoresis by conventional means. Polypeptides can be transferred to a suitable protein binding membrane, such as nitrocellulose, by conventional means.

Polypeptides on the membrane can be visualized using two different methods that allow a discrimination between the sample protein and the molecular weight markers. H14 polypeptide or fragmented peptide molecular weight markers can be visualized using antibodies generated against these markers and conventional immunoblotting techniques. This detection is performed under conventional conditions that do not result in the detection of the sample protein. It is understood that it may not be possible to generate antibodies against all H14 polypeptide fragments, since small peptides may not contain immunogenic epitopes. It is further understood that not all antibodies will work in this assay; however, those antibodies which are able to bind H14 polypeptides and fragments can be readily determined using conventional techniques.

The sample protein is visualized using a conventional staining procedure. The molar excess of sample protein to H14 polypeptide or fragmented peptide molecular weight markers is such that the conventional staining procedure predominantly detects the sample protein. The level of H14 polypeptide or fragmented peptide molecular weight markers is such as to allow little or no detection of these markers by the conventional staining method. The preferred molar excess of sample protein to H14 polypeptide molecular weight markers is between 2 and 100,000 fold. More preferably, the preferred molar excess of sample protein to H14 polypeptide molecular weight markers is between 10 and 10,000 fold and especially between 100 and 1,000 fold.

The H14 polypeptide or fragmented peptide molecular weight markers can be used as molecular weight and isoelectric point markers in the estimation of the apparent molecular weight and isoelectric point of the sample protein. The H14 polypeptide or fragmented peptide molecular weight markers serve particularly well as molecular weight and isoelectric point markers for the estimation of apparent molecular weights and isoelectric points of sample proteins that have apparent molecular weights and isoelectric points close to that of the H14 polypeptide or fragmented peptide molecular weight markers. The ability to simultaneously resolve the H14 polypeptide or fragmented peptide molecular weight markers and the sample protein under identical conditions allows for increased accuracy in the determination of the apparent molecular weight and isoelectric point of the sample protein. This is of particular interest in techniques, such as two dimensional electrophoresis, where the nature of the procedure dictates that any markers should be resolved simultaneously with the sample protein.

In another embodiment, H14 polypeptide or fragmented peptide molecular weight markers can be used as molecular weight and isoelectric point markers in the estimation of the apparent molecular weight and isoelectric point of fragmented peptides derived by treatment of a sample protein with a cleavage agent. It is understood of course that many techniques can be used for the determination of molecular weight and isoelectric point of a sample protein and fragmented peptides thereof using H14 polypeptide molecular weight markers and peptide fragments thereof and that this embodiment in no way limits the scope of the invention.

H14 polypeptide molecular weight markers encompassed by invention can have variable molecular weights, depending upon the host cell in which they are expressed. Glycosylation of H14 polypeptide molecular weight markers and peptide fragments thereof in various cell types can result in variations of the molecular weight of these markers, depending upon the extent of modification. The size of H14 polypeptide molecular weight markers can be most heterogeneous with fragments of H14 polypeptide derived from the extracellular portion of the polypeptide. Consistent molecular weight markers can be obtained by using polypeptides derived entirely from the transmembrane and cytoplasmic regions, pretreating with N-glycanase to remove glycosylation, or expressing the polypeptides in bacterial hosts.

The interaction between H14 and its counter-structure enables screening for small molecules that interfere with the H14/H14 counter-structure association and inhibit activity of H14 or its counter-structure. For example, the yeast two-hybrid system developed at SUNY (described in U.S. Pat. No. 5,283,173 to Fields et al.) can be used to screen for inhibitors of H14 as follows. H14 and its counter-structure, or portions thereof responsible for their interaction, can be fused to the Gal4 DNA binding domain and Gal 4 transcriptional activation domain, respectively, and introduced into a strain that depends on Gal4 activity for growth on plates lacking histidine. Compounds that prevent growth can be screened in order to identify IL-1 inhibitors. Alternatively, the screen can be modified so that H14/H14 counter-structure interaction inhibits growth, so that inhibition of the interaction allows growth to occur. Another, in vitro, approach to screening for H14 inhibition would be to immobilize one of the components (either H14 or its counter-structure) in wells of a microtiter plate, and to couple an easily detected indicator to the other component. An inhibitor of the interaction is identified by the absence of the detectable indicator from the well.

In addition, H14 polypeptides according to the invention are useful for the structure-based design of an H14 inhibitor. Such a design would comprise the steps of determining the three-dimensional structure of such the H14 polypeptide, analyzing the three-dimensional structure for the likely binding sites of substrates, synthesizing a molecule that incorporates a predictive reactive site, and determining the inhibiting activity of the molecule.

Antibodies immunoreactive with H14 polypeptides, and in particular, monoclonal antibodies against H14 polypeptides, are now made available through the invention. Such antibodies can be useful for inhibiting H14 polypeptide activity in vivo and for detecting the presence of H14 polypeptides in a sample.

As used herein, the term “H14 polypeptides” refers to a genus of polypeptides that further encompasses proteins having the amino acid sequence 1-426 of SEQ ID NO: 2, as well as those proteins having a high degree of similarity (at least 90% homology) with such amino acid sequences and which proteins are biologically active. In addition, H14 polypeptides refers to the gene products of the nucleotides 1-1281 of SEQ ID NO: 1.

The isolated and purified H14 polypeptide according to the invention has a molecular weight of approximately 46,896 Daltons. It is understood that the molecular weight of H14 polypeptides can be varied by fusing additional peptide sequences to both the amino and carboxyl terminal ends of H14 polypeptides. Fusions of additional peptide sequences at the amino and carboxyl terminal ends of H14 polypeptides can be used to enhance expression of H14 polypeptides or aid in the purification of the protein.

It is understood that fusions of additional peptide sequences at the amino and carboxyl terminal ends of H14 polypeptides will alter some, but usually not all, of the fragmented peptides of H14 polypeptides generated by enzymatic or chemical treatment.

It is understood that mutations can be introduced into H14 polypeptides using routine and known techniques of molecular biology. It is further understood that a mutation can be designed so as to eliminate a site of proteolytic cleavage by a specific enzyme or a site of cleavage by a specific chemically induced fragmentation procedure. It is also understood that the elimination of the site will alter the peptide fingerprint of H14 polypeptides upon fragmentation with the specific enzyme or chemical procedure.

The term “isolated and purified” as used herein, means that the H14 polypeptide molecular weight markers or fragments thereof are essentially free of association with other proteins or polypeptides, for example, as a purification product of recombinant host cell culture or as a purified product from a non-recombinant source. The term “substantially purified” as used herein, refers to a mixture that contains H14 polypeptide molecular weight markers or fragments thereof and is essentially free of association with other proteins or polypeptides, but for the presence of known proteins that can be removed using a specific antibody, and which substantially purified H14 polypeptides or fragments thereof can be used as molecular weight markers. The term “purified” refers to either the “isolated and purified” form of H14 polypeptides or the “substantially purified” form of H14 polypeptides, as both are described herein.

A “nucleotide sequence” refers to a polynucleotide molecule in the form of a separate fragment or as a component of a larger nucleic acid construct, that has been derived from DNA or RNA isolated at least once in substantially pure form (i.e., free of contaminating endogenous materials) and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

An H14 polypeptide “variant” as referred to herein means a polypeptide substantially homologous to native H14 polypeptides, but which has an amino acid sequence different from that of native H14 polypeptides (human, murine or other mammalian species) because of one or more deletions, insertions or substitutions. The variant amino acid sequence preferably is at least 80% identical to a native H14 polypeptide amino acid sequence, most preferably at least 90% identical. The percent identity can be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring H14 variants are also encompassed by the invention. Examples of such variants are proteins that result from alternate mRNA splicing events or from proteolytic cleavage of the H14 polypeptides. Variations attributable to proteolysis include, for example, differences in the N- or C-termini upon expression in different types of host cells, due to proteolytic removal of one or more terminal amino acids from the H14 polypeptides (generally from 1-5 terminal amino acids).

As stated above, the invention provides isolated and purified, or homogeneous, H14 polypeptides, both recombinant and non-recombinant. Variants and derivatives of native H14 polypeptides that can be used as molecular weight markers can be obtained by mutations of nucleotide sequences coding for native H14 polypeptides. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making the alterations set forth above are disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

H14 polypeptides can be modified to create H14 polypeptide derivatives by forming covalent or aggregative conjugates with other chemical moieties, such as glycosyl groups, polyethylene glycol (PEG) groups, lipids, phosphate, acetyl groups and the like. Covalent derivatives of H14 polypeptides can be prepared by linking the chemical moieties to functional groups on H14 polypeptide amino acid side chains or at the N-terminus or C-terminus of a H14 polypeptide or the extracellular domain thereof. Other derivatives of H14 polypeptides within the scope of this invention include covalent or aggregative conjugates of H14 polypeptides or peptide fragments with other proteins or polypeptides, such as by synthesis in recombinant culture as N-terminal or C-terminal fusions. For example, the conjugate can comprise a signal or leader polypeptide sequence (e.g. the α-factor leader of Saccharomyces) at the N-terminus of a H14 polypeptide. The signal or leader peptide co-translationally or post-translationally directs transfer of the conjugate from its site of synthesis to a site inside or outside of the cell membrane or cell wall.

H14 polypeptide conjugates can comprise peptides added to facilitate purification and identification of H14 polypeptides. Such peptides include, for example, poly-His or the antigenic identification peptides described in U.S. Pat. No. 5,011,912 and in Hopp et al., Bio/Technology 6:1204, 1988.

The invention further includes H14 polypeptides with or without associated native-pattern glycosylation. H14 polypeptides expressed in yeast or mammalian expression systems (e.g., COS-1 or COS-7 cells) can be similar to or significantly different from a native H14 polypeptide in molecular weight and glycosylation pattern, depending upon the choice of expression system. Expression of H14 polypeptides in bacterial expression systems, such as E. coli, provides non-glycosylated molecules. Glycosyl groups can be removed through conventional methods, in particular those utilizing glycopeptidase. In general, glycosylated H14 polypeptides can be incubated with a molar excess of glycopeptidase (Boehringer Mannheim).

Equivalent DNA constructs that encode various additions or substitutions of amino acid residues or sequences, or deletions of terminal or internal residues or sequences are encompassed by the invention. For example, N-glycosylation sites in the H14 polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn—X—Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846, hereby incorporated by reference.

In another example, sequences encoding Cys residues that are not essential for biological activity can be altered to cause the Cys residues to be deleted or replaced with other amino acids, preventing formation of incorrect intramolecular disulfide bridges upon renaturation. Other equivalents are prepared by modification of adjacent dibasic amino acid residues to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding, or substituting residues to alter Arg—Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

The invention further encompasses isolated fragments and oligonucleotides derived from the nucleotide sequence of SEQ ID NO: 1, including nucleotide sequence 1-697 of SEQ ID NO: 1, nucleotide sequence 698-923 of SEQ ID NO: 1, and nucleotide sequence 924-1278 of SEQ ID NO: 1. The invention also encompasses polypeptides encoded by these fragments and oligonucleotides.

Nucleic acid sequences within the scope of the invention include isolated DNA and RNA sequences that hybridize to the native H14 nucleotide sequences disclosed herein under conditions of moderate or severe stringency, and which encode H14 polypeptides. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of about 60° C., 0.5×SSC, 0.1% SDS. Conditions of high stringency are defined as hybridization conditions as above, and with washing at 68° C., 0.2×SSC, 0.1% SDS. The skilled artisan will recognize that the temperature and wash solution salt concentration can be adjusted as necessary according to factors such as the length of the probe.

Due to the known degeneracy of the genetic code wherein more than one codon can encode the same amino acid, a DNA sequence can vary from that shown in SEQ ID NO: 1 and still encode a H14 polypeptide having the amino acid sequence of SEQ ID NO: 2. Such variant DNA sequences can result from silent mutations (e.g., occurring during PCR amplification), or can be the product of deliberate mutagenesis of a native sequence.

The invention thus provides equivalent isolated DNA sequences encoding H14 polypeptides, selected from: (a) DNA derived from the coding region of a native mammalian H14 gene; (b) cDNA comprising the nucleotide sequence 1-1281 of SEQ ID NO: 1; (c) DNA capable of hybridization to a DNA of (a) under conditions of moderate stringency and which encodes H14 polypeptides; and (d) DNA which is degenerate as a result of the genetic code to a DNA defined in (a), (b) or (c) and which encodes H14 polypeptides. H14 polypeptides encoded by such DNA equivalent sequences are encompassed by the invention.

DNA that is equivalent to the DNA sequence of SEQ ID NO: 1 will hybridize under moderately stringent conditions to the double-stranded native DNA sequence that encode polypeptides comprising amino acid sequences of 1-426 of SEQ ID NO: 2. Examples of H14 polypeptides encoded by such DNA, include, but are not limited to, H14 polypeptide fragments and H14 polypeptides comprising inactivated N-glycosylation site(s), inactivated protease processing site(s), or conservative amino acid substitution(s), as described above. H14 polypeptides encoded by DNA derived from other mammalian species, wherein the DNA will hybridize to the complement of the DNA of SEQ ID NO: 1 are also encompassed.

H14 polypeptide-binding proteins, such as the anti-H14 polypeptide antibodies of the invention, can be bound to a solid phase such as a column chromatography matrix or a similar substrate suitable for identifying, separating or purifying cells that express H14 polypeptides on their surface. Adherence of H14 polypeptide-binding proteins to a solid phase contacting surface can be accomplished by any means, for example, magnetic microspheres can be coated with H14 polypeptide-binding proteins and held in the incubation vessel through a magnetic field. Suspensions of cell mixtures are contacted with the solid phase that has H14 polypeptide-binding proteins thereon. Cells having H14 polypeptides on their surface bind to the fixed H14 polypeptide-binding protein and unbound cells then are washed away. This affinity-binding method is useful for purifying, screening or separating such H14 polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner.

Alternatively, mixtures of cells suspected of containing H14 polypeptide-expressing cells first can be incubated with a biotinylated H14 polypeptide-binding protein. Incubation periods are typically at least one hour in duration to ensure sufficient binding to H14 polypeptides. The resulting mixture then is passed through a column packed with avidin-coated beads, whereby the high affinity of biotin for avidin provides the binding of the H14 polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

In the methods described above, suitable H14 polypeptide-binding proteins are anti-H14 polypeptide antibodies, and other proteins that are capable of high-affinity binding of H14 polypeptides. A preferred H14 polypeptide-binding protein is an anti-H14 polypeptide monoclonal antibody.

H14 polypeptides can exist as oligomers, such as covalently linked or non-covalently linked dimers or trimers. Oligomers can be linked by disulfide bonds formed between cysteine residues on different H14 polypeptides. In one embodiment of the invention, a H14 polypeptide dimer is created by fusing H14 polypeptides to the Fc region of an antibody (e.g., IgG1) in a manner that does not interfere with biological activity of H14 polypeptides. The Fc polypeptide preferably is fused to the C-terminus of a soluble H14 polypeptide (comprising only the extracellular domain). General preparation of fusion proteins comprising heterologous polypeptides fused to various portions of antibody-derived polypeptides (including the Fc domain) has been described, e.g., by Ashkenazi et al. (PNAS USA 88:10535, 1991) and Byrn et al. (Nature 344:677, 1990), hereby incorporated by reference. A gene fusion encoding the H14 polypeptide:Fc fusion protein is inserted into an appropriate expression vector. H14 polypeptide:Fc fusion proteins are allowed to assemble much like antibody molecules, whereupon interchain disulfide bonds form between Fc polypeptides, yielding divalent H14 polypeptides. If fusion proteins are made with both heavy and light chains of an antibody, it is possible to form a H14 polypeptide oligomer with as many as four H14 polypeptides extracellular regions. Alternatively, one can link two soluble H14 polypeptide domains with a peptide linker.

Recombinant expression vectors containing a nucleic acid sequence encoding H14 polypeptides can be prepared using well known methods. The expression vectors include a H14 DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the H14 DNA sequence. Thus, a promoter nucleotide sequence is operably linked to a H14 DNA sequence if the promoter nucleotide sequence controls the transcription of the H14 DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with H14 polypeptides can be incorporated into expression vectors. For example, a DNA sequence for a signal peptide (secretory leader) can be fused in-frame to the H14 nucleotide sequence so that the H14 polypeptide is initially translated as a fusion protein comprising the signal peptide. A signal peptide that is functional in the intended host cells enhances extracellular secretion of the H14 polypeptide. The signal peptide can be cleaved from the H14 polypeptide upon secretion of H14 polypeptide from the cell.

Suitable host cells for expression of H14 polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce H14 polypeptides using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a H14 polypeptide can include an N-terminal methionine residue to facilitate expression of the recombinant polypeptide in the prokaryotic host cell. The N-terminal Met can be cleaved from the expressed recombinant H14 polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a H14 DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA). Other commercially available vectors include those that are specifically designed for the expression of proteins; these would include pMAL-p2 and pMAL-c2 vectors that are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982). A particularly useful prokaryotic host cell expression system employs a phage λ P_{L} promoter and a cI857ts thermolabile repressor sequence. Plasmid vectors available from the American Type Culture Collection, which incorporate derivatives of the λ P_{L} promoter, include plasmid pHUB2 (resident in E. coli strain JMB9 (ATCC 37092)) and pPLc28 (resident in E. coli RR1 (ATCC 53082)).

H14 DNA may be cloned in-frame into the multiple cloning site of an ordinary bacterial expression vector. Ideally the vector would contain an inducible promoter upstream of the cloning site, such that addition of an inducer leads to high-level production of the recombinant protein at a time of the investigator's choosing. For some proteins, expression levels may be boosted by incorporation of codons encoding a fusion partner (such as hexahistidine) between the promoter and the gene of interest. The resulting “expression plasmid” may be propagated in a variety of strains of E. coli.

For expression of the recombinant protein, the bacterial cells are propagated in growth medium until reaching a pre-determined optical density. Expression of the recombinant protein is then induced, e.g. by addition of IPTG (isopropyl-b-D-thiogalactopyranoside), which activates expression of proteins from plasmids containing a lac operator/promoter. After induction (typically for 1-4 hours), the cells are harvested by pelleting in a centrifuge, e.g. at 5,000×G for 20 minutes at 4° C.

For recovery of the expressed protein, the pelleted cells may be resuspended in ten volumes of 50 mM Tris-HCl (pH 8)/1 M NaCl and then passed two or three times through a French press. Most highly-expressed recombinant proteins form insoluble aggregates known as inclusion bodies. Inclusion bodies can be purified away from the soluble proteins by pelleting in a centrifuge at 5,000×G for 20 minutes, 4° C. The inclusion body pellet is washed with 50 mM Tris-HCl (pH 8)/1% Triton X-100 and then dissolved in 50 mM Tris-HCl (pH 8)/8 M urea/0.1 M DTT. Any material that cannot be dissolved is removed by centrifugation (10,000×G for 20 minutes, 20° C.). The protein of interest will, in most cases, be the most abundant protein in the resulting clarified supernatant. This protein may be “refolded” into the active conformation by dialysis against 50 mM Tris-HCl (pH 8)/5 mM CaCl₂/5 mM Zn(OAc)₂/1 mM GSSG/0.1 mM GSH. After refolding, purification can be carried out by a variety of chromatographic methods such as ion exchange or gel filtration. In some protocols, initial purification may be carried out before refolding. As an example, hexahistidine-tagged fusion proteins may be partially purified on immobilized Nickel.

While the preceding purification and refolding procedure assumes that the protein is best recovered from inclusion bodies, those skilled in the art of protein purification will appreciate that many recombinant proteins are best purified out of the soluble fraction of cell lysates. In these cases, refolding is often not required, and purification by standard chromatographic methods can be carried out directly.

H14 polypeptides alternatively can be expressed in yeast host cells, preferably from the Saccharomyces genus (e.g., S. cerevisiae). Other genera of yeast, such as Pichia, K. lactis, or Kluyveromyces, can also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980), or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli can be constructed by inserting DNA sequences from pBR322 for selection and replication in E. coli (Amp^{r} gene and origin of replication) into the above-described yeast vectors.

The yeast α-factor leader sequence can be employed to direct secretion of a H14 polypeptide. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U. S. Pat. No. 4,546,082; and EP 324,274. Other leader sequences suitable for facilitating secretion of recombinant polypeptides from yeast hosts are known to those of skill in the art. A leader sequence can be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 μg/ml adenine, and 20 μg/ml uracil.

Yeast host cells transformed by vectors containing ADH2 promoter sequence can be grown for inducing expression in a “rich” medium. An example of a rich medium is one consisting of 1% yeast extract, 2% peptone, and 1% glucose supplemented with 80 μg/ml adenine and 80 μg/ml uracil. Derepression of the ADH2 promoter occurs when glucose is exhausted from the medium.

Mammalian or insect host cell culture systems could also be employed to express recombinant H14 polypeptides. Baculovirus systems for production of heterologous proteins in insect cells are reviewed by Luckow and Summers, Bio/Technology 6:47 (1988). Established cell lines of mammalian origin also can be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line (ATCC CRL 10478) derived from the African green monkey kidney cell line CVI (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine (Gibco/BRL) or Lipofectamine-Plus, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. Molecular Cloning: A Laboratory Manual, 2 ed. Vol. 1-3, Cold Spring Harbor Laboratory Press, 1989). Selection of stable transformants can be performed using resistance to cytotoxic drugs as a selection method. Kaufman et al., Meth. in Enzymology 185:487-511, 1990, describes several selection schemes, such as dihydrofolate reductase (DHFR) resistance. A suitable host strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (eg. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in U.S. patent application Ser. No. 07/701,415, filed May 16, 1991, incorporated by reference herein. The vectors can be derived from retroviruses. In place of the native signal sequence, a heterologous signal sequence can be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type H IL-1 receptor signal peptide described in EP 460,846.

An isolated and purified H14 polypeptide molecular weight marker according to the invention can be produced by recombinant expression systems as described above or purified from naturally occurring cells. H14 polypeptides can be substantially purified, as indicated by a single protein band upon analysis by SDS-polyacrylamide gel electrophoresis (SDS-PAGE).

One process for producing H14 polypeptides comprises culturing a host cell transformed with an expression vector comprising a DNA sequence that encodes a H14 polypeptide under conditions sufficient to promote expression of the H14 polypeptide. H14 polypeptide is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify H14 polypeptides. Some or all of the foregoing purification steps, in various combinations, are well known and can be employed to provide an isolated and purified recombinant protein.

It is possible to utilize an affinity column comprising a H14 polypeptide-binding protein, such as a monoclonal antibody generated against H14 polypeptides, to affinity-purify expressed H14 polypeptides. H14 polypeptides can be removed from an affinity column using conventional techniques, e.g., in a high salt elution buffer and then dialyzed into a lower salt buffer for use or by changing pH or other components depending on the affinity matrix utilized.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells are preferably employed to express H14 polypeptides as secreted polypeptides in order to simplify purification. Secreted recombinant polypeptide from a yeast host cell fermentation can be purified by methods analogous to those disclosed by Urdal et al. (J. Chromatog. 296:171, 1984). Urdal et al. describe two sequential, reversed-phase HPLC steps for purification of recombinant human IL-2 on a preparative HPLC column.

H14 polypeptide molecular weight markers can be analyzed by methods including sedimentation, gel electrophoresis, chromatography, and mass spectrometry. H14 polypeptides can serve as molecular weight markers using such analysis techniques to assist in the determination of the molecular weight of a sample protein. A molecular weight determination of the sample protein assists in the identification of the sample protein.

H14 polypeptides can be subjected to fragmentation into peptides by chemical and enzymatic means. Chemical fragmentation includes the use of cyanogen bromide to cleave under neutral or acidic conditions such that specific cleavage occurs at methionine residues (E. Gross, Methods in Enz. 11:238-255, 1967). This can further include further steps, such as a carboxymethylation step to convert cysteine residues to an unreactive species. Enzymatic fragmentation includes the use of a protease such as Asparaginylendopeptidase, Arginylendopeptidase, Achrombobacter protease I, Trypsin, Staphlococcus aureus V8 protease, Endoproteinase Asp-N, or Endoproteinase Lys-C under conventional conditions to result in cleavage at specific amino acid residues. Asparaginylendopeptidase can cleave specifically on the carboxyl side of the asparagine residues present within H14 polypeptides. Arginylendopeptidase can cleave specifically on the carboxyl side of the arginine residues present within H14 polypeptides. Achrombobacter protease I can cleave specifically on the carboxyl side of the lysine residues present within H14 polypeptides (Sakiyama and Nakat, U.S. Pat. No. 5,248,599; T. Masaki et al., Biochim. Biophys. Acta 660:44-50, 1981; T. Masaki et al., Biochim. Biophys. Acta 660:51-55, 1981). Trypsin can cleave specifically on the carboxyl side of the arginine and lysine residues present within H14 polypeptides. Staphlococcus aureus V8 protease can cleave specifically on the carboxyl side of the aspartic and glutamic acid residues present within H14 polypeptides (D. W. Cleveland, J. Biol. Chem. 3:1102-1106, 1977). Endoproteinase Asp-N can cleave specifically on the amino side of the asparagine residues present within H14 polypeptides. Endoproteinase Lys-C can cleave specifically on the carboxyl side of the lysine residues present within H14 polypeptides. Other enzymatic and chemical treatments can likewise be used to specifically fragment H14 polypeptides into a unique set of specific peptide molecular weight markers.

The resultant fragmented peptides can be analyzed by methods including sedimentation, electrophoresis, chromatograpy, and mass spectrometry. The fragmented peptides derived from H14 polypeptides can serve as molecular weight markers using such analysis techniques to assist in the determination of the molecular weight of a sample protein. Such a molecular weight determination assists in the identification of the sample protein. H14 fragmented peptide molecular weight markers are preferably between 10 and 425 amino acids in size. More preferably, H14 fragmented peptide molecular weight markers are between 10 and 100 amino acids in size. Even more preferable are H14 fragmented peptide molecular weight markers between 10 and 50 amino acids in size and especially between 10 and 35 amino acids in size. Most preferable are H14 fragmented peptide molecular weight markers between 10 and 20 amino acids in size.

Furthermore, analysis of the progressive fragmentation of H14 polypeptides into specific peptides (D. W. Cleveland et al., J. Biol. Chem. 252:1102-1106, 1977), such as by altering the time or temperature of the fragmentation reaction, can be used as a control for the extent of cleavage of a sample protein. For example, cleavage of the same amount of H14 polypeptide and sample protein under identical conditions can allow for a direct comparison of the extent of fragmentation. Conditions that result in the complete fragmentation of H14 polypeptide can also result in complete fragmentation of the sample protein.

In addition, H14 polypeptides and fragmented peptides thereof possess unique charge characteristics and, therefore, can serve as specific markers to assist in the determination of the isoelectric point of a sample protein or fragmented peptide using techniques such as isoelectric focusing. The technique of isoelectric focusing can be further combined with other techniques such as gel electrophoresis to simultaneously separate a protein on the basis of molecular weight and charge. An example of such a combination is that of two-dimensional electrophoresis (T. D. Brock and M. T. Madigan, Biology of Microorganisms 76-77 (Prentice Hall, 6d ed. 1991)). H14 polypeptides and fragmented peptides thereof can be used in such analyses as markers to assist in the determination of both the isoelectric point and molecular weight of a sample protein or fragmented peptide.

Kits to aid in the determination of apparent molecular weight and isoelectric point of a sample protein can be assembled from H14 polypeptides and peptide fragments thereof. Kits also serve to assess the degree of fragmentation of a sample protein. The constituents of such kits can be varied, but typically contain H14 polypeptide and fragmented peptide molecular weight markers. Also, such kits can contain H14 polypeptides wherein a site necessary for fragmentation has been removed. Furthermore, the kits can contain reagents for the specific cleavage of H14 and the sample protein by chemical or enzymatic cleavage. Kits can further contain antibodies directed against H14 polypeptides or fragments thereof.

Antisense or sense oligonucleotides comprising a single-stranded nucleic acid sequence (either RNA or DNA) capable of binding to a target H14 mRNA sequence (forming a duplex) or to the H14 sequence in the double-stranded DNA helix (forming a triple helix) can be made according to the invention. Antisense or sense oligonucleotides, according to the present invention, comprise a fragment of the coding region of H14 cDNA (SEQ ID NO: 1). Such a fragment generally comprises at least about 14 nucleotides, preferably from about 14 to about 30 nucleotides. The ability to create an antisense or a sense oligonucleotide, based upon a cDNA sequence for a given protein is described in, for example, Stein and Cohen, Cancer Res. 48:2659, 1988 and van der Krol et al., BioTechniques 6:958, 1988.

Binding of antisense or sense oligonucleotides to target nucleic acid sequences results in the formation of complexes that block translation (RNA) or transcription (DNA) by one of several means, including enhanced degradation of the duplexes, premature termination of transcription or translation, or by other means. The antisense oligonucleotides thus can be used to block expression of H14 polypeptides. Antisense or sense oligonucleotides further comprise oligonucleotides having modified sugar-phosphodiester backbones (or other sugar linkages, such as those described in WO91/06629), and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences. Other examples of sense or antisense oligonucleotides include those oligonucleotides that are covalently linked to organic moieties, such as those described in WO 90/10448, and other moieties that increase affinity of the oligonucleotide for a target nucleic acid sequence, such as poly-(L-lysine). Further still, intercalating agents, such as ellipticine, and alkylating agents or metal complexes can be attached to sense or antisense oligonucleotides to modify binding specificities of the antisense or sense oligonucleotide for the target nucleotide sequence.

Antisense or sense oligonucleotides can be introduced into a cell containing the target nucleic acid sequence by any gene transfer method, including, for example, CaPO₄-mediated DNA transfection, electroporation, or by using gene transfer vectors such as Epstein-Barr virus. Antisense or sense oligonucleotides are preferably introduced into a cell containing the target nucleic acid sequence by insertion of the antisense or sense oligonucleotide into a suitable retroviral vector, then contacting the cell with the retrovirus vector containing the inserted sequence, either in vivo or ex vivo. Suitable retroviral vectors include, but are not limited to, the murine retrovirus M-MuLV, N2 (a retrovirus derived from M-MuLV), or the double copy vectors designated DCT5A, DCT5B and DCT5C (see PCT Application US 90/02656).

Sense or antisense oligonucleotides also can be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide can be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Isolated and purified H14 polypeptides or a fragment thereof can also be useful itself as a therapeutic agent in inhibiting TNF signaling. H14 polypeptides are introduced into the intracellular environment by well-known means, such as by encasing the protein in liposomes or coupling it to a monoclonal antibody targeted to a specific cell type.

H14 DNA, H14 polypeptides, and antibodies against H14 polypeptides can be used as reagents in a variety of research protocols. A sample of such research protocols are given in Sambrook et al. Molecular Cloning: A Laboratory Manual, 2 ed. Vol. 1-3, Cold Spring Harbor Laboratory Press, (1989). For example, these reagents can serve as markers for cell specific or tissue specific expression of RNA or proteins. Similarly, these reagents can be used to investigate constituitive and transient expression of H14 RNA or polypeptides. H14 DNA can be used to determine the chromosomal location of H14 DNA and to map genes in relation to this chromosomal location. H14 DNA can also be used to examine genetic heterogeneity and heredity through the use of techniques such as genetic fingerprinting, as well as to identify risks associated with genetic disorders. H14 DNA can be further used to identify additional genes related to H14 DNA and to establish evolutionary trees based on the comparison of sequences. H14 DNA and polypeptides can be used to select for those genes or proteins that are homologous to H14 DNA or polypeptides, through positive screening procedures such as Southern blotting and immunoblotting and through negative screening procedures such as subtraction.

H14 polypeptides can also be used as a reagent to identify (a) any protein that H14 polypeptide regulates, and (b) other proteins with which it might interact. H14 polypeptides could be used by coupling recombinant protein to an affinity matrix, or by using them as a bait in the 2-hybrid system.

When used as a therapeutic agent, H14 polypeptides can be formulated into pharmaceutical compositions according to known methods. H14 polypeptides can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain H14 polypeptides complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of H14 polypeptides.

Within an aspect of the invention, H14 polypeptides, and peptides based on the amino acid sequence of H14, can be utilized to prepare antibodies that specifically bind to H14 polypeptides. The term “antibodies” is meant to include polyclonal antibodies, monoclonal antibodies, fragments thereof such as F(ab′)2, and Fab fragments, as well as any recombinantly produced binding partners. Antibodies are defined to be specifically binding if they bind H14 polypeptides with a Kₐ of greater than or equal to about 10⁷ M⁻¹. Affinities of binding partners or antibodies can be readily determined using conventional techniques, for example those described by Scatchard et al., Ann. N.Y Acad. Sci., 51:660 (1949).

Polyclonal antibodies can be readily generated from a variety of sources, for example, horses, cows, goats, sheep, dogs, chickens, rabbits, mice, or rats, using procedures that are well-known in the art. In general, purified H14 polypeptides, or a peptide based on the amino acid sequence of H14 polypeptides that is appropriately conjugated, is administered to the host animal typically through parenteral injection. The immunogenicity of H14 polypeptides can be enhanced through the use of an adjuvant, for example, Freund's complete or incomplete adjuvant. Following booster immunizations, small samples of serum are collected and tested for reactivity to H14 polypeptides. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radio-immunoprecipitation, enzyme-linked immuno-sorbent assays (ELISA), dot blot assays, and sandwich assays, see U.S. Pat. Nos. 4,376,110 and 4,486,530.

Monoclonal antibodies can be readily prepared using well-known procedures, see for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980. Briefly, the host animals, such as mice are injected intraperitoneally at least once, and preferably at least twice at about 3 week intervals with isolated and purified H14 polypeptides or conjugated H14 polypeptides, optionally in the presence of adjuvant. Mouse sera are then assayed by conventional dot blot technique or antibody capture (ABC) to determine which animal is best to fuse. Approximately two to three weeks later, the mice are given an intravenous boost of H14 polypeptides or conjugated H14 polypeptides. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as, ¹²⁵I—H14 polypeptides is added to each well followed by incubation. Positive wells can be subsequently detected by autoradiography. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Other types of “antibodies” can be produced using the information provided herein in conjunction with the state of knowledge in the art. For example, antibodies that have been engineered to contain elements of human antibodies that are capable of specifically binding H14 polypeptides are also encompassed by the invention.

Once isolated and purified, the antibodies against H14 polypeptides can be used to detect the presence of H14 polypeptides in a sample using established assay protocols. Further, the antibodies of the invention can be used therapeutically to bind to H14 polypeptides and inhibit its activity in vivo.

The purified H14 polypeptides according to the invention will facilitate the discovery of inhibitors of H14 polypeptides. The use of a purified H14 polypeptide in the screening of potential inhibitors thereof is important and can eliminate or reduce the possibility of interfering reactions with contaminants.

In addition, H14 polypeptides can be used for structure-based design of H14 polypeptide-inhibitors. Such structure-based design is also known as “rational drug design.” The H14 polypeptides can be three-dimensionally analyzed by, for example, X-ray crystallography, nuclear magnetic resonance or homology modeling, all of which are well-known methods. The use of H14 polypeptide structural information in molecular modeling software systems to assist in inhibitor design and inhibitor-H14 polypeptide interaction is also encompassed by the invention. Such computer-assisted modeling and drug design can utilize information such as chemical conformational analysis, electrostatic potential of the molecules, protein folding, etc. For example, most of the design of class-specific inhibitors of metalloproteases has focused on attempts to chelate or bind the catalytic zinc atom. Synthetic inhibitors are usually designed to contain a negatively-charged moiety to which is attached a series of other groups designed to fit the specificity pockets of the particular protease. A particular method of the invention comprises analyzing the three dimensional structure of H14 polypeptides for likely binding sites of substrates, synthesizing a new molecule that incorporates a predictive reactive site, and assaying the new molecule as described above.

The specification is most thoroughly understood in light of the teachings of the references cited within the specification, which are hereby incorporated by reference. The embodiments within the specification provide an illustration of embodiments of the invention and should not be construed to limit the scope of the invention. The skilled artisan recognizes many other embodiments are encompassed by the claimed invention.

What is claimed is:

1. (Three times amended) An isolated nucleic acid molecule selected from the group consisting of:
(a) an isolated nucleic acid molecule comprising the DNA sequence of SEQ ID NO: 1;
(b) an isolated nucleic acid molecule encoding an amino acid sequence comprising the sequence of SEQ ID NO: 2; and
(c) an isolated nucleic acid molecule that hybridizes to a nucleic acid comprising the nucleic acid sequence of (a) or (b) in 50% formamide and 6×SSC at 42° C. with washing conditions of 60° C., 0.5×SSC, 0.1% SDS, wherein said nucleic acid molecule encodes a polypeptide comprising amino acids 135-345 of SEQ ID NO: 2.

2. A recombinant vector that directs the expression of a nucleic acid molecule of claim 1.

8. (Amended) A host cell comprising the vector of claim 2.

9. (Amended) A method for the production of H14 polypeptide comprising culturing a host cell of claim 8 under conditions promoting expression.

10. The method of claim 9, wherein the host cell is selected from the group consisting of bacterial cells, yeast cells, plant cells, and animal cells.

17. (Amended) A fusion polypeptide comprising amino acids 32-339 of SEQ ID NO: 3 fused to heterologous amino acids.

18. The fusion polypeptide of claim 17, wherein said heterologous amino acids comprise the Fc region of an antibody and a CMV leader.

19 The fusion polypeptide of claim 18 comprising amino acids 1-544 of SEQ ID NO: 3.

20. The fusion polypeptide of claim 19 comprising amino acids 21-544 of SEQ ID NO: 3.

21. (Two times amended) An isolated nucleic acid molecule selected from:
(a) an isolated nucleic acid molecule encoding an amino acid sequence comprising the sequence of SEQ ID NO: 3;
(b) an isolated nucleic acid molecule encoding an amino acid sequence comprising amino acids 32-339 of SEQ ID NO: 3; and
(c) an isolated nucleic acid molecule encoding an amino acid sequence comprising amino acids 21-339 of SEQ ID NO: 3.

23. (Amended) An isolated nucleic acid molecule comprising a DNA sequence selected from the group consisting of:
(a) SEQ ID NO: 1;
(b) nucleotides 1-1278 of SEQ ID NO: 1;
(c) nucleotides 1-697 of SEQ ID NO: 1; and
(d) nucleotides 698-923 of SEQ ID NO: 1.

24. An isolated nucleic acid molecule encoding an amino acid sequence comprising amino acids 1-426 of SEQ ID NO: 2.

25. (Amended) An isolated nucleic acid molecule encoding a polypeptide comprising amino acids 135-345 of SEQ ID NO: 2.

26. (Amended) An isolated nucleic acid molecule encoding a polypeptide comprising amino acids 37-134 of SEQ ID NO: 2.

27. (Amended) An isolated nucleic acid molecule encoding an amino acid sequence selected from the group consisting of:
(a) amino acids 1 to 314 of SEQ ID NO: 2;
(b) amino acids 1 to 345 of SEQ ID NO: 2;
(c) an amino acid sequence starting at any one of amino acids 31-37 and terminating at amino acid 134 of SEQ ID NO: 2;
(d) an amino acid sequence starting at any one of amino acids 31-37 and terminating at amino acid 345 of SEQ ID NO: 2; and
(e) an amino acid sequence starting at any one of amino acids 31-37 and terminating at amino acid 426 of SEQ ID NO: 2.

28. (Amended) An isolated nucleic acid molecule encoding an amino acid sequence selected from the group consisting of:
(a) amino acids 1-544 of SEQ ID NO: 3;
(b) amino acids 21-544 of SEQ ID NO: 3;
(c) amino acids 32-544 of SEQ ID NO: 3;
(d) amino acids 1-339 of SEQ ID NO: 3;
(e) amino acids 21-339 of SEQ ID NO: 3; and
(f) amino acids 32-339 of SEQ ID NO: 3.


SEQUENCE LISTING (3 sequences)

SEQ ID NO: 1 - DNA, 1281 bp, Homo sapiens
atgcccgccggccgccggggccccgccgcccaatccgcgcggcggccgccgccgttgctgcccctgctgctgctgctctgcgtcctcggggcgccgcgagccggatcaggagcccacacagctgtgatcagtccccaggatcccacgcttctcatcggctcctccctgctggccacctgctcagtgcacggagacccaccaggagccaccgccgagggcctctactggaccctcaacgggcgccgcctgccccctgagctctcccgtgtactcaacgcctccaccttggctctggccctggccaacctcaatgggtccaggcagcggtcgggggacaacctcgtgtgccacgcccgtgacggcagcatcctggctggctcctgcctctatgttggcctgcccccagagaaacccgtcaacatcagctgctggtccaagaacatgaaggacttgacctgccgctggacgccaggggcccacggggagaccttcctccacaccaactactccctcaagtacaagcttaggtggtatggccaggacaacacatgtgaggagtaccacacagtggggccccactcctgccacatccccaaggacctggctctctttacgccctatgagatctgggtggaggccaccaaccgcctgggctctgcccgctccgatgtactcacgctggatatcctggatgtggtgaccacggaccccccgcccgacgtgcacgtgagccgcgtcgggggcctggaggaccagctgagcgtgcgctgggtgtcgccacccgccctcaaggatttcctctttcaagccaaataccagatccgctaccgagtggaggacagtgtggactggaaggtggtggacgatgtgagcaaccagacctcctgccgcctggccggcctgaaacccggcaccgtgtacttcgtgcaagtgcgctgcaacccctttggcatctatggctccaagaaagccgggatctggagtgagtggagccaccccacagccgcctccactccccgcagtgagcgcccgggcccgggcggcggggcgtgcgaaccgcggggcggagagccgagctcggggccggtgcggcgcgagctcaagcagttcctgggctggctcaagaagcacgcgtactgctccaacctcagcttccgcctctacgaccagtggcgagcctggatgcagaagtcgcacaagacccgcaaccagcacaggacgaggggatcctgccctcgggcagacggggcacggcgagaggtcctgccagataagctgtag

SEQ ID NO: 2 - protein, 426 aa, Homo sapiens
MPAGRRGPAAQSARRPPPLLPLLLLLCVLGAPRAGSGAHTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQHRTRGSCPRADGARREVLPDKL

SEQ ID NO: 3 - protein, 544 aa, Artificial Sequence
  Description of Artificial Sequence Fusion protein
MARRLWILSLLAVTLTVALAAPSQKSKRRTSGAHTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPLGYRYVEPRSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESKGQPENNYKTTPPVLDSDGYTQKSLSLSPGK